(19) United States
(12) Patent Application Publication
(10) Pub. No.: US 2007/0238630 A1
(43) Pub. Date: Oct. 11, 2007

(54) Subcutaneous skin cleanser

(76) Inventor: Douglas Howard Phillips, Millerton, OK (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/392,171

(22) Filed: Mar. 29, 2006

(51) Int. Cl. A61K 8/00

(52) U.S. Cl. 510/130

(57) ABSTRACT

An anti-venom and anti-toxin subcutaneous skin cleanser applied topically to a bite, sting or other skin irritation to quickly relieve the associated itching and irritation. The cleanser is a combination of surfactant chemicals that are known to increase the porosity and/or permeation of the skin, chemicals which can dissolve venoms and toxins into water-based fluids, chemicals that can cause gentle abrasion for the purpose of opening up the pathway from the skin surface, and chemicals which are chemically active in a way that they can change the molecular structure of the venom or toxin.

### BACKGROUND OF THE INVENTION

1. Field of the Invention

The present invention relates to a form of soap that cleanses the skin, specifically to regions of tissue that are below the surface of the skin. It is useful in cleansing the skin following exposure to poison ivy or following insect bites.

2. Description of the Related Art

This cleanser invention uses anionic surfactants, i.e., surface reactant chemicals. The defining feature of the anionic surfactant is that it is an anion, i.e., a negatively-charged ion. Some general-purpose soaps contain anionic surfactants.

Specifically, this cleanser invention uses the anionic surfactant sodium dodecylbenzenesulfonate which is available from most chemical suppliers. Synonyms for this surfactant include sodium dodecylbenzenesulfonate, benzenesulfonic acid, and dodecylbenzenesulfonic acid.

One aspect of the novelty of this cleanser invention is in the surprisingly successful results obtained on poison ivy rash through the actual use of this cleanser. An excellent result, including 100% success in more than 100 trials, has been obtained using this invention to treat poison ivy and eliminate the itching in less than one minute.

The word “toxin” as used in this document refers to non-animal-produced substances harmful to humans. Examples are urushiol oil from poison ivy and other plant-produced chemicals and/or industry-produced chemicals which come into contact with human skin.

The word “venom” as used in this document refers to animal-produced substances harmful to humans. Examples are venom from insect bites and/or stings, snake bites, fish bites and/or stings and other animal-produced harmful substances which come into contact with human skin.

The word “dissolve” as used in this document refers to the action of a surfactant chemical which causes certain oils to mix with water-based substances. This mixing results in an “apparent dissolving” of the oil into the water-based substance.

Near-100% effectiveness was achieved when this invention was used as a poison ivy cleanser to dry up weeping lesions. Most cleansers and other forms of treatment for poison ivy are much less than 100% effective for humans. This is also true for other kinds of treatments, including prescription drugs and home remedies. Most remedies of any kind are considered commercially viable if they are successful more than about 60% of the time. One objective of this cleanser invention is to be virtually 100% effective when used to remove urushiol oil toxic effects, specifically including drying up the weeping lesions on the skin.

This invention is fast acting, within one minute, when used as a poison ivy cleanser. One objective of this cleanser invention is to be effective very quickly, virtually instantly, when used to stop the itching on the skin of humans suffering from poison ivy exposure. This is important because when the itching stops, the scratching stops, thereby limiting the spread of the urushiol oil and the spreading of inflammation to nearby areas of the skin. As a fast-acting solution, most common cleansers and other forms of treatment for poison ivy are much less than 100% effective for all humans. The objective of this cleanser invention is to be virtually 100% effective, when used to remove urushiol oil toxic effects, specifically including stopping the itching on the skin, within one minute of the application as a skin cleanser.

This invention is effective and fast-acting when used as an anti-venom. An objective of this cleanser invention is to be effective when used to stop the swelling and itching, of the skin, of humans suffering from bites and stings from insects and other non-human animals.

This invention results in synergistic benefits because of the use of more than one active chemical ingredient. An objective of this invention is to combine commonly-used chemicals to achieve greater effectiveness than could be achieved by any one of the chemicals if used alone.

This invention uses commercially available common chemicals. An objective of this invention is to have it comprise only chemicals that are available from many chemical suppliers. A consideration for any new cleanser invention is whether it will be accepted for use by the general public. A related objective of this invention was to use chemicals that are available as part of many commercially-available household products.

This invention is safe for human use. An objective of this invention is to use the advantage of safety-of-use, to make this cleanser invention a practical one. This cleanser invention uses chemicals in concentrations currently approved and sold as cleansers for skin-contact limited use.

This invention can be manufactured easily. An objective of this invention was to manufacture it using simple, low-cost methods that do not require special handling because of chemical instability or other critical factors.

The primary regulatory advantage of this cleanser invention is that this invention is subject to Federal Trade Commission, or FTC, requirements and guidelines instead of the Federal Drug Administration or FDA. This is an advantage because research and development costs are less for many reasons, including the ability to commercialize this cleanser invention without the need for filing a new drug application with the FDA. This cleanser invention is a form of soap that functions as a skin cleanser. It is not a drug.

A second regulatory advantage is its safety-of-use which makes this cleanser invention a practical one. This cleanser invention uses chemicals in concentrations currently approved and sold as cleansers for skin-contact limited use. This cleanser invention, unlike many “new use inventions” has easily been made available commercially. Beginning in September 2005, this cleanser invention has been used successfully in field trials and commercial trials, when used both as an anti-toxin, for treating the effects of poison ivy; and when used as an anti-venom, for treating the effects of insect bites and stings.

One practical advantage of this invention is that it functions as an anti-allergen to provide cleansing action which destroys and/or modifies and/or dissolves a toxin or allergen that has penetrated the skin and has become a subcutaneous source of harmful effects.

A second practical advantage of this invention is that it functions as an anti-venom to provide cleansing action which destroys and/or modifies and/or dissolves a venom that has been injected into the skin, or has penetrated the skin because of the porous nature of the skin, and has become a subcutaneous source of harmful effects.

A third practical advantage of this invention is that it provides effectiveness through the use of synergy and the combination of four effects; dissolving oil-based substances, improving chemical transport via skin permeation, improved chemical transport via gentle skin abrasion and broadening the chemical arsenal.

Dissolving is one of four ways in which this invention achieves effectiveness. Because many toxins and venoms are oil-based substances, this cleanser invention uses a surfactant chemical which can dissolve certain oils, including toxins and venoms, into water-based fluids so that the body can more-easily dissipate and process the harmless by-products of the dissolved remnants of the toxins and venoms.

A second way in which this invention achieves effectiveness is the use of skin permeation to improve chemical transport: Because it is difficult for topically-applied cleansers to physically contact a subcutaneously-located toxin or venom, this cleanser invention uses a surfactant chemical that is known to increase the porosity and permeation of the skin because of the temporary effect on the pores of the skin. In this way, the surfactant helps to provide a pathway from the skin surface, where the cleanser is topically applied, to the physical location of the subcutaneously-located toxin or venom.

Anionic surfactants are commonly used in household products. They are used in shampoos, in dishwashing detergents and in washing powders. Long-term human exposure to anionic surfactants has been linked to swelling of the skin in a conditioned allergic reaction. This swelling is temporary, although it tends to increase the susceptibility of the skin to permeation by the surfactant and by other substances. Anionic surfactants are generally avoided in cosmetic products, but their use in shampoos and other products can still lead to irritation. That is why changing shampoos every month or so is considered a good idea by many people.

One non-obvious aspect of this cleanser invention is that, for this subcutaneous cleanser, the increase in permeation of the skin is used to advantage; and is not a disadvantage for the new-use applications described in this patent application. As described later in this document, long-term use is not required to achieve the beneficial effects of this cleanser invention.

A third way in which this invention achieves effectiveness is the use of gentle abrasion to increase chemical transport. This invention is intended to provide a cleansing benefit when the skin has been harmed by a puncture wound, caused by a sting or bite in the case of attacks by venomous insects, snakes and other animals; or a lesion such as an open, weeping rash in the case of poison ivy and similar irritants. This cleanser invention uses gentle abrasion as a method to open up the pathway from the skin surface, where the cleanser is topically applied, to the physical location of the subcutaneously-located toxin or venom.

This invention uses chemically-passive, almost chemically inert, gritty additives so that when this cleanser invention is rubbed onto the skin, the scrubbing can open up the pathway for use in chemical transport of the cleanser to the allergen or toxin. This cleanser invention uses carbonates, in the preferred embodiment, although other gritty substances could provide for gentle abrasion just as well as the carbonates that have been used in one specific preferred embodiment.

A fourth way in which this invention achieves effectiveness is by broadening the chemical arsenal. This invention uses hypochlorite bleaching agents containing chlorine, in the form of weak bleach, to chemically attack the toxin or venom. Chlorine is one of the most chemically-active elements available and allowed, for safety reasons, where skin contact is involved. This cleanser invention uses chlorine to supplement the chemical attack provided by the surfactant. Chlorine is not absolutely necessary for this cleanser invention to work well. The purpose of the chlorine is to provide chemical activity, specifically the ability to attack and combine with oil molecules. Therefore, this invention broadens the chemical arsenal used for effective cleansing in this cleanser invention.

The preferred embodiment uses chlorine for reasons described above. An obvious alternative embodiment would be to eliminate the use of chlorine or to substitute another chemically-active substance such as ammonia.

A fourth practical advantage of this invention is that it uses surfactant chemistry. Many, but not all detergents and soaps are classified as surfactants. The name “surfactant” stands for surface active agent. A surfactant molecule has a hydrophilic, water-loving head. A surfactant molecule also has a long hydrophobic, water-hating or oil-loving, tail. Surfactant molecules are said to be amphiphilic molecules because they seem to easily combine with both water-based substances and oil-based substances.

The properties of surfactants fall into two broad categories: adsorption and self-assembly. Adsorption is the tendency for a surfactant molecule to collect at an interface and can sometimes become the interface between an oil and a water-based substance. An oil may be a toxin or venom in the case of this cleanser invention. A water-based substance may be body tissue or body fluids in the case of this cleanser invention. Absorption is the taking up of one substance by another. Adsorption is very different from absorption.

The adsorption properties of surfactants mean that surfactant molecules are usually found at the interface between an oil phase and a water phase or a water phase and an air phase. This molecular property leads to the macroscopic properties of wetting, foaming, detergency and emulsion formation.

Surfactant molecules tend to adsorb to the surface of oil droplets. The hydrophilic heads stick out into the water phase, while the hydrophobic tails stick into the oil phase. The result is that a surfactant can cause certain oils to mix with water-based substances. This mixing results in an “apparent dissolving” of the oil into the water-based substance. This process is what is meant in this document when the terms “dissolve, dissolved or dissolving” are used.

### SUMMARY OF THE INVENTION

This invention relates to a soap for cleansing the skin, specifically regions of tissue that are below the surface of the skin.

Common skin abnormalities result from subcutaneous toxins from plants, chemicals or venom injected from stings and bites. When a toxin lies beneath the surface of the skin, it is usually difficult to modify, remove or destroy.

An example of subcutaneous toxins from plants is urushiol oil from poison ivy, poison oak and poison sumac. When the surface of the skin is exposed to urushiol oil, the pores in the skin provide a pathway for the toxin to penetrate the skin and become a subcutaneous toxin.

Examples of subcutaneous toxins from non-human species include toxins that are injected or deposited from bites and stings. Ants, wasps, some snakes, some fish and other species have the ability to deposit venom in this fashion.

Some skin cleansers rely on washing and removing the toxin from the skin. That method is often not completely effective, as in the case of poison ivy, because the toxin is an oil-based chemical which does not respond to water-based skin cleansers.

This cleanser invention relies on a more effective method. The objective of this cleanser invention is to chemically attack and modify and/or destroy the toxin, thereby rendering it harmless as a toxin. To accomplish this objective, the skin cleanser must make chemical and physical contact with the toxin.

Because subcutaneous toxins are covered by the top layer of skin, it is difficult to bring the cleanser chemical into direct chemical and physical contact with the toxin. This cleanser invention solves this problem by using both chemical and physical methods.

Evaluation conducted during the development of this invention resulted in the conclusion that the surfactant, when used at a minimum concentration of approximately 1% to 2% is entirely adequate and that range of concentrations is what has been used to manufacture this cleanser invention. This surfactant concentration is less than what is used in most existing and previously-approved commercially-available household products that contain sodium dodecylbenzenesulfonate.

In the preferred embodiment of this cleanser invention, sodium dodecylbenzenesulfonate has been used as the surface reactive anion agent. Additional chemicals are mixed with this surfactant to broaden the chemical spectrum used in the attack of the toxin or venom; to provide a gritty texture; and to adjust the color and fragrance of the mixture.

Calcium carbonate, approximately 1% to 80% by weight, is used for abrasive and dilation. Hypochlorite bleaching agents, approximately 1% to 10% by weight, is used to attack the allergen. Sodium carbonate, approximately 1% to 80% by weight, is used for abrasive and dilation. Surface reactive anion agents, approximately 1% to 2% by weight, are used for skin permeation and to dissolve the allergen. Cocoa bean powder, approximately 1% to 30% by weight, is used as a binder and for color. Talc powder, approximately 1% to 30% by weight, is used to provide a desired fragrance and as a binder.

The weight percentage of each ingredient is varied to optimize the mixture for specific applications. This is illustrated with two examples as follows.

In the first example, if the mixture is intended to be used as a poison ivy remedy, the mixture will be left on the skin for a few days. For this application, the coloring agent weight percentage is adjusted to have a skin-tone color.

In another example, if the mixture is intended to be used as a remedy for insect stings, the mixture will be washed away immediately after only one application. For this application of the mixture, there is no need for a coloring agent and the weight percentage can be reduced, thereby providing a stronger (less dilute) mixture for the intended application.

Similar considerations can lead to the formulation that is more or less gritty, thereby resulting in a greater or lesser weight percentage in the mixture of sodium carbonate and calcium carbonate.

All chemicals used in this mixture are available from most chemical suppliers. All of these chemicals are in general use for common commercial and household products.

Concentrations of the chemicals can be varied widely and this invention can still be effective. After the chemicals are added to a hopper, stirring is used to achieve a near-homogenous mixture. After the vials or containers are filled with the powder mixture, a mechanical vibrator is used to achieve some compaction of the powder so that lids/stoppers/caps can be used to complete the packaging of the cleanser product.

Directions for anti-allergen use of this invention are as follow: Wet finger tip with water, or saliva, if in the field, and dip tip of finger in powder. When using saliva, care must be taken to NOT spread the urushiol oil from the poison ivy to the mouth. After mixing the powder with a source of moisture, the resulting gritty paste provides a mild abrasive action. Rub the paste into the skin where itching results from poison oak bumps and blisters that are “weeping.” These lesions are said to be weeping when there is evidence of clear liquid being produced and oozing from the affected skin. Rub the paste into the skin for 30 seconds or until itching stops. For best results, leave paste on skin and allow it to dry into a film. The film will continue to combat the effects of the poison oak and/or poison ivy. Wash with running water and reapply as itching occurs. Use as needed; overuse is not a problem. Long-term use, beyond 3 days, is not recommended or required to achieve the beneficial effects of this cleanser invention.

Similar directions for use apply to use of this cleanser invention as an anti-venom skin cleanser. The effectiveness of this invention has been tested for the stings of ants and yellow-jackets; and the bites of mosquitoes. At the time of this writing, this invention has not yet been tested for snakebite, although limited testing is planned in the future.

Almost always, a sting or bite is followed by swelling of the skin. The swelling tends to close off the puncture-wound channel that can be used to physically transport the topically-applied cleanser to the subcutaneous venom. Consequently, directions for use of this cleanser must include a requirement to apply the cleanser within minutes after a person has sustained a sting or bite. The reason is that this cleanser works as a contact anti-venom, meaning that the cleanser must come in chemical/physical contact with the venom so that beneficial effects can be obtained.

### DETAILED DESCRIPTION OF THE PREFERRED EMBODIMENT THE INVENTION

This cleanser invention relates to cleansing the skin, specifically to regions of tissue that are below the surface of the skin. Some skin cleansers rely on washing and removing the toxin from the skin.

This cleanser invention chemically attacks the toxin below the skin surface. It employs both chemical and physical agents to bring the cleanser into direct chemical and physical contact with a subcutaneous toxin. Those ingredients are:

| Active Ingredients | Purpose |
|---|---|
| Calcium carbonate (1-80%)* | Abrasive and dilation |
| Hypochlorite bleaching agents (1-10%)* | Anti-allergen |
| Sodium carbonate (1-80%)* | Abrasive and dilation |
| Surface reactive anion agents (1-2%)* | Skin permeation and |
| | anti-allergen |

*In concentrations currently approved and sold as cleansers for skin-contact limited use but not as skin cleansers. The concentrations above are given in units of weight percentage.

The preferred embodiment of this cleanser invention uses the following inactive ingredients in addition to the ingredients listed and described above:

| Inactive Ingredients | Purpose |
|---|---|
| Coloring agent such as | Binder and color |
| Cocoa bean powder (1-30%)* | |
| Fragrance agent such as | Fragrance and binder |
| Talc powder (1-30%)* | |

*In concentrations currently approved and sold as cleansers for skin-contact limited use but not as skin cleansers. The concentrations above are given in units of weight percentage.

The chemicals used in this cleanser invention are available from many chemical suppliers. Also, these chemicals are available as part of many commercially-available household products.

In the preferred embodiment of this cleanser invention, sodium dodecylbenzenesulfonate has been used as the surface reactive anion agent. This chemical is available from most chemical suppliers, and is in general use for common commercial products.

All chemicals listed above are obtained in powder form from commercial sources. Weight percent has been used as the measure when mixing the chemicals. There is no particular order, based on chemical reaction considerations, which is required during mlixing.

Evaluation conducted during the development of this invention resulted in the conclusion that the surfactant, when used at a minimum concentration of approximately 1% to 2% is entirely adequate and that range of concentrations is what has been used to manufacture this cleanser invention. This surfactant concentration is less than what is used in most existing and previously-approved commercially-available household products that contain sodium dodecylbenzenesulfonate.

Concentrations of the chemicals can be varied widely and this invention can still be effective. Preparing a paste is the preferred method for application of the cleanser to the skin. The sodium carbonate and the calcium carbonate mixing percentage is maintained adequate to achieve the goal of having a slightly gritty mixture when a thick paste is made using water. The hypochlorite bleaching agents are included with a mixing percentage that is common for household uses including scrubbing and washing. The cocoa bean powder mixing percentage is varied to achieve a skin-tone color for the product, applied to a weeping lesion in powder form or when mixed with water, at the time of application, to form a thick paste. The talc powder mixing percentage is adjusted to provide a pleasing scent.

After the chemicals are added to a hopper, stirring is used to achieve a near-homogenous mixture. The existence of the color additive makes visual observation an easy method to determine when the mixture is nearly-homogenous.

After the vials or containers are filled with the powder mixture, a mechanical vibrator is used to achieve some compaction of the powder so that lids/stoppers/caps can be used to complete the packaging of the cleanser product.

Preparing a paste, by mixing water with the powder, is the preferred method for application of the cleanser to the skin. An obvious alternative embodiment would be to use a source of moisture other than water to prepare the paste. Any non-oily source of moisture, such as alcohol, can be used to prepare the paste.

An obvious alternative embodiment would be to use different additives, such as common food coloring agents, to achieve the desired color.

An obvious alternative embodiment would be to use different additives, such as common cosmetic fragrance agents, to achieve the desired fragrance.

### Use of the Invention

Wet finger tip with water, or saliva, if in the field, and dip tip of finger in powder. When using saliva, care must be taken to not spread the urushiol oil from the poison ivy to the mouth. After mixing the powder with a source of moisture, the resulting gritty paste provides a mild abrasive action. Rub the paste into the skin where itching results from poison oak bumps and blisters that are “weeping.” These lesions are said to be weeping when there is evidence of clear liquid being produced and oozing from the affected skin. Rub the paste into the skin for 30 seconds or until itching stops. For best results, leave paste on skin and allow it to dry into a film. The film will continue to combat the effects of the poison oak and/or poison ivy. Wash with running water and reapply as itching occurs. Use as needed; overuse is not a problem. Long-term use, beyond 3 days, is not recommended or required to achieve the beneficial effects of this cleanser invention.

To cleanse sting wounds and bite wounds, the cleanser is used in the same manner as described above. The effectiveness of this invention has been tested for the stings of ants and yellow-jackets; and the bites of mosquitoes. This invention has not yet been tested for snakebite, although limited testing is planned in the future.

Almost always, a sting or bite is followed by swelling, which tends to close off the puncture-wound channel that can be used to physically transport the applied cleanser to the subcutaneous venom. Consequently, directions for use of this cleanser must include a requirement to apply the cleanser within minutes after a person has sustained a sting or bite. The reason is that this cleanser works as a contact anti-venom, meaning that the cleanser must come in chemical/physical contact with the venom so that beneficial effects can be obtained. Soon after a bite or sting, the affected skin will often begin to swell. As explained above, this swelling tends to close off the puncture-wound channel that can be used to physically transport the applied cleanser to the subcutaneous venom.

Although the invention has been described as a powder, it is not so limited, and could be formulated as a liquid or a gel.

The use of water, saliva or another liquid is used as a convenient transfer mechanism to get the cleanser from a container to the lesion on the skin. An obvious alternative method of use would be to use the cleanser without water, by applying the powder directly to the lesion.

The preferred embodiment features the use of this cleanser invention for treating common skin abnormalities that result from subcutaneous toxins because when a toxin lies beneath the surface of the skin, it is usually more difficult to remove or destroy. An obvious alternative method of use would be to use this cleanser invention to cleanse the surface of the skin.

An obvious alternative use would be to use this invention for a wider range of allergic reactions including, for example, the so-called jewelry allergy that sometimes results from wearing jewelry of various materials in close contact with the skin.

Although the invention has been illustrated by certain of the preceding alternative embodiments and methods of use, it is not to be construed as being limited thereby; but rather, the invention encompasses the generic area as hereinbefore disclosed. Various modifications and embodiments can be made without departing from the spirit and scope thereof.

What is claimed is:

1. An anti-allergen and anti-toxin subcutaneous skin cleanser comprising:
one or more surfactant chemicals that are known to increase the porosity and/or permeation of the skin, and one or more chemicals which can dissolve toxins and venoms into water-based fluids, and one or more chemicals which are chemically active in a way that said chemicals can change the molecular structure of toxins and venoms.

2. A skin cleanser according to claim 1 wherein the surfactant chemicals are selected from the following list: The anionic surfactant sodium dodecylbenzenesulfonate, sodium dodecylbenzenesulfonate, benzenesulfonic acid, and dodecylbenzenesulfonic acid.

3. A skin cleanser according to claim 2 wherein the chemicals which can dissolve toxins and venoms into water-based fluids are selected from the following list: sodium dodecylbenzenesulfonate, benzenesulfonic acid, and dodecylbenzenesulfonic acid.

4. A skin cleanser according to claim 3 wherein the chemicals which are chemically active in a way that said chemicals can change the molecular structure of toxins and venoms are selected from the following list: sodium hypochlorite, calcium hypochlorite, magnesium hypochlorite, potassium hypochlorite, manganese hypochlorite, iron hypochlorite, tin hypochlorite, and gold hypochlorite.

5. An anti-allergen and anti-toxin subcutaneous skin cleanser according to claim 4, further comprising one or more chemicals that are gritty when mixed with fluid and topically applied to the skin, and can cause gentle abrasion for the purpose of opening up the pathway from the skin surface to the physical location of the subcutaneously-located toxin and/or venom.

6. A skin cleanser according to claim 5 wherein the chemicals that are gritty when mixed with fluid are selected from the following list: calcium carbonate and sodium carbonate.
